(19) United States
(12) Patent Application Publication
(10) Pub. No.: US 2022/0142890 A1
(43) Pub. Date: May 12, 2022

(54) AQUEOUS DISPERSION OF A COSMETIC PIGMENT AND A METHOD FOR PREPARING THE SAME

(71) Applicant: NISSIN CHEMICAL INDUSTRY CO., LTD., Echizen-shi, Fukui (JP)

(72) Inventor: Kentaro WATANABE, Echizen-shi (JP)

(73) Assignee: NISSIN CHEMICAL INDUSTRY CO., LTD., Echizen-shi, Fukui (JP)

(21) Appl. No.: 17/430,862

(22) PCT Filed: Feb. 10, 2020
(86) PCT No.: PCT/JP2020/005045

§ 371 Date: Aug. 13, 2021

(30) Foreign Application Priority Data
Feb. 15, 2019 (JP) 2019-025591

(51) Int. Cl. A61K 8/58; A61K 8/04; A61K 8/73; A61K 8/29; A61K 8/27; A61K 8/26; A61Q 17/04

(52) U.S. Cl. CPC A61K 8/585; A61K 8/04; A61K 8/731; A61K 8/29; A61K 2800/612; A61K 8/26; A61Q 17/04; A61K 2800/43; A61K 8/27

(57) ABSTRACT

One of the purposes of the present invention is to provide an aqueous dispersion comprising a cosmetic pigment, which aqueous dispersion is stable with time, capable of forming a film with sufficient water resistance (water repellency), and provides a stable cosmetic preparation which does not cause caking; and a method for preparing the aqueous dispersion. The present invention provides an aqueous dispersion of a cosmetic pigment, water and hydroxypropylmethyl cellulose phthalate, wherein the cosmetic pigment is surface-silylated with an alkyltrialkoxysilane. The present invention further provides a method for preparing an aqueous dispersion containing the cosmetic pigment having a surface-silylated with an alkyltrialkoxysilane.

### TECHNICAL FIELD

The present invention relates to an aqueous dispersion of a cosmetic pigment and a method for preparing the same.

### BACKGROUND OF THE INVENTION

Inorganic UV absorbents such as titanium oxide and zinc oxide are well known to have a higher UV absorption performance, compared to organic UV absorbents. Inorganic UV absorbents are less stimulating and less toxic and give less burden to human bodies and environments, which are advantageous. Therefore, they are used, for instance, in sunscreen creams. Inorganic pigments such as ultramarine and titanium mica are used, for instance, in eyeliners. Inorganic UV absorbents are less stimulating to the skin and less harmful to human bodies, compared to organic pigments. Therefore, they are very useful as a cosmetic pigment.

However, it is difficult to disperse inorganic pigments, such as titanium oxide and zinc oxide, in water. This is because they have higher specific gravities than water, so that they likely to precipitate in water. If their surface is hydrophobized, they may easily cause aggregation in water. In many cases, inorganic pigments are used in combination with a surfactant or a water-soluble polymer in order to enhance dispersion in water. However, this may frequently cause such an inconvenience that a refreshing and non-sticky feel of touch characteristic of water-based cosmetics may not be obtained. In addition, a large amount of a surfactant or water-soluble polymer is often required to provide an aqueous dispersion stable with time. Then, water-resistance is almost hopeless.

For example, Japanese Patent Application Laid-Open No. Hei 10-251125 (Patent Literature 1) describes that various water-soluble polymers are used as a dispersant to have titanium oxide dispersed in water. However, the water-soluble polymers impede water resistance and a refreshing and non-sticky feel of touch characteristic of water-based cosmetics on a skin. Therefore, it is difficult to use them for cosmetics.

Various proposals have been made to overcome the above-described problems. For example, Japanese Patent Application Laid-Open No. 2008-150328 (Patent Literature 2) describes that a feel of touch is improved by decreasing an amount of a water-soluble polymer, wherein an aqueous dispersion of hydrated silica-treated titanium oxide is used. A film formed from the aqueous dispersion does not exhibit water repellency, so that it has a problem in water resistance. Besides, an amount of the water-soluble polymer added is small, so that there is still has a problem in storage stability.

Japanese Patent Application Laid-Open No. 2015-105257 (Patent Literature 3) describes that a stable aqueous dispersion is obtained by modifying the surface of titanium oxide with a polyglycerin chain and adding sodium chloride. A film obtained from the aqueous dispersion does not have sufficient water resistance.

Japanese Patent Application Laid-Open No. 2007-224050 (Patent Literature 4) describes an aqueous dispersion of talc or titanium mica as a cosmetic composition. The aqueous dispersion comprises a solvent selected from the group consisting of relatively polar, nonvolatile oils and non-polar, nonvolatile oils (such as cyclomethicone), as a main component, so that a refreshing and non-sticky feel characteristic of water-based cosmetics cannot be achieved. Besides, the aqueous dispersion comprises a polyoxyalkylene unit, so that a film obtained from the dispersion is inferior in water resistance.

Japanese National Phase Publication No. Hei 8-505624 (Patent Literature 5) describes an oil-in-water type emulsion composition which comprises dispersed titanium oxide, zinc oxide or ultramarine, which are surface-hydrophobized with silicone, and a cosmetic thereof. The composition has a premise that it contains a large amount of a humectant such as urea. When the composition is a sunscreen cosmetic, sufficient water resistance cannot be achieved.

### PRIOR LITERATURES

### Patent Literatures

Patent Literature 1: Japanese Patent Application Laid-Open No. Hei 10-251125
Patent Literature 2: Japanese Patent Application Laid-Open No. 2008-150328
Patent Literature 3: Japanese Patent Application Laid-Open No. 2015-105257
Patent Literature 4: Japanese Patent Application Laid-Open No. 2007-224050
Patent Literature 5: Japanese National Phase Publication No. Hei 8-505624

### SUMMARY OF THE INVENTION

### Problems to be Solved by the Invention

One of the purposes of the present invention is to provide an aqueous dispersion of a cosmetic pigment, which aqueous dispersion is stable with time, capable of forming a film with sufficient water resistance (water repellency), and provides a stable cosmetic preparation which does not cause caking; and a method for preparing the aqueous dispersion.

### Means for Solving the Problems

The present inventor conducted keen researches to solve the aforesaid problems and have found that an aqueous dispersion which has excellent dispersion stability so as not to cause aggregation for a long period of time and gives a film having good water resistance is obtained by silylating a cosmetic pigment with an alkyltrialkoxysilane to obtain a surface-silylated cosmetic pigment and incorporating hydroxypropylmethyl cellulose phthalate as a dispersant in an aqueous dispersion of the pigment.

That is, the present invention provides an aqueous dispersion comprising a cosmetic pigment, water and hydroxypropylmethyl cellulose phthalate, wherein the cosmetic pigment is surface-silylated with an alkyltrialkoxysilane. Preferably the cosmetic pigment is at least one selected from the group consisting of titanium oxide, zinc oxide, ultramarine, and titanium mica. The present invention further provides a method for preparing an aqueous dispersion containing the cosmetic pigment having a surface-silylated with an alkyltrialkoxysilane.

### Effects of the Invention

The aqueous dispersion of the present invention is excellent in dispersion stability and gives excellent water resistance to a film obtained from the dispersion. Further, the aqueous dispersion provides a cosmetic which shows less change, such as caking, with time, so that the dispersion is suitable for use in cosmetics.

### DETAILED DESCRIPTION OF THE INVENTION

The present invention relates to an aqueous dispersion of a cosmetic pigment and a method for preparing the same. One of the characteristics of the aqueous dispersion is that a surface of the cosmetic pigment is silylated with alkyltrialkoxysilane. The surface silylation of the cosmetic pigment suppresses aggregation of the cosmetic pigment to provide an aqueous dispersion having long-term stability. Further, the method for preparing an aqueous dispersion containing the surface-silylated cosmetic pigment is characterized in that hydroxypropylmethyl cellulose phthalate is incorporated as a dispersant in the aqueous dispersion.

Hydroxypropylmethyl cellulose phthalate functions effectively to disperse the cosmetic pigment in water. Hydroxypropylmethyl cellulose phthalate is neutralized with an acid such as citric acid after the silylation reaction. The aqueous dispersion thus treated has improved water repellency, so that a liquid cosmetic containing the aqueous dispersion forms a coating film having good water resistance or water repellency. Further, the liquid cosmetic containing the hydroxypropylmethyl cellulose phthalate can be easily removed with a soap.

The cosmetic pigment to be contained in the aqueous dispersion of the present invention may be any known cosmetic pigment in a form of powder or particle. Examples of the cosmetic pigment include inorganic pigments, organic pigments, and composite pigments. Preferred examples include inorganic pigments such as extender pigments, color pigments, white pigments, and pearlescent pigments. More specific examples include titanium oxide, zinc oxide, ultramarine, titanium mica, Prussian blue, red oxide, yellow oxide, aluminum oxide, cerium oxide, silicic anhydride, magnesium oxide, zirconium oxide, magnesium carbonate, calcium carbonate, chromium oxide, chromium hydroxide, carbon black, aluminum silicate, magnesium silicate, magnesium aluminum silicate, mica, synthetic mica, synthetic sericite, sericite, talc, kaolin, silicon carbide, barium sulfate, bentonite, smectite, and boron nitride. However, Black iron oxide is not preferred. Of these, titanium oxide, zinc oxide, ultramarine, and titanium mica are particularly preferred. They may be surface-treated with alumina, silica or a water-soluble polymer.

Examples of the titanium oxide include STR-100 (not surface-treated), STR-100C (treated with alumina), and STR-100W (treated with silica), all ex Sakai Chemical Industry Co., Ltd.; and MT-500B (untreated), MT-100AQ (sodium alginate), and MT-100SA (silica, alumina), all ex TAYCA CO., LTD.

Examples of the zinc oxide include FINEX-50M (not surface-treated) and FINEX-30M (hydrated silica), all ex Sakai Chemical Industry Co.,Ltd., and XZ-100F-LP and MZ-500, all ex TAYCA CO., LTD.

Examples of the ultramarine include 17, Ultramarine TR (ex VENATOR), and Ultramarine Blue (ex Fujifilm Wako Pure Chemical Corporation).

Examples of the titanium mica include Timiron Super Red (ex Merck) and FANTASPEARL 1060T-WR, FANTASPEARL 1060T-GA, PROMINENCE SF, and PROMINENCE RF, all ex Nihon Koken Kogyo Co., Ltd.

The cosmetic pigment is preferably in a fine particle form. Titanium oxide and zinc oxide preferably have an average particle size of from 1 to 100 nm, more preferably from 10 to 50 nm, from the standpoint of a chromogenic property and ultraviolet omission. Ultramarine and titanium mica preferably have an average particle size of from 1 to 100 μm, more preferably from 2 to 70 μm. In the present invention, the average particle size is determined by the laser diffraction/scattering method. The particle shape of the cosmetic pigment is not particularly limited and may be, for example, a needle shape or spherical shape, as long as it has the aforesaid average particle size.

The aqueous dispersion of the present invention contains the surface-silylated cosmetic pigment in an amount of 1 to 30 mass % as the cosmetic pigment. The amount of the cosmetic pigment does not include the amount of the silyl moiety present on the surface of the cosmetic pigment. The amount of the cosmetic pigment in the aqueous dispersion is from 1 to 30 mass %, preferably from 10 to 27 mass %, more preferably from 15 to 23 mass %, based on the total mass of the aqueous dispersion. The amount within this range ensures good dispersion stability.

The alkyltrialkoxysilane is known as a silylating agent. The alkyl group may have 1 to 10 carbon atoms, preferably 1 to 5 carbon atoms. The alkoxy group may have 1 to 5 carbon atoms, preferably 1 to 3 carbon atoms. Examples of the alkyltrialkoxysilane include methyltriethoxysilane, methyltrimethoxysilane, propyltriethoxysilane, octyltriethoxysilane, and ethyltriethoxysilane. Among these, alkyltriethoxysilanes are highly reactive with the cosmetic pigment and the byproduced ethanol is safe and easily distilled off. Therefore, the alkyltriethoxysilanes are easy to handle as a silylating agent for the cosmetic pigment and are preferred.

The amount of the alkyltrialkoxysilane used for the silylation may be from 0.1 to 20 parts by mass, preferably from 0.5 to 15 parts by mass, more preferably from 1 to 13 parts by mass, relative to 100 parts by mass of the cosmetic pigment. By silylating the surface of the cosmetic pigment in an amount of the aforesaid range, aggregation of the cosmetic pigment is effectively suppressed and an aqueous dispersion having long-term stability is provided. If the amount of the alkyltrialkoxysilane is less than the aforesaid lower limit, the aforesaid effects are not attained sufficiently. If the amount of the alkyltrialkoxysilane is larger than the aforesaid upper limit, aggregation of the particles may occur, so that color development of a cosmetic may be poor. Almost all of the alkyltrialkoxysilane in an amount within the aforesaid range adheres to the surface of the cosmetic pigment in the silylation. However, the aqueous dispersion of the present invention may contain a free alkyltrialkoxysilane without participating in the silylation. In the aqueous dispersion of the present invention, therefore, the amount of the alkyltrialkoxysilane may be from 0.5 to 20 parts by mass, preferably from 1 to 15 parts by mass, more preferably from 3 to 10 parts by mass, relative to 100 parts by mass of the cosmetic pigment.

The viscosity of the hydroxypropylmethyl cellulose phthalate may be selected properly, preferably from 10 to 200 mPa·s, more preferably from 10 to 100 mPa·s, at 20° C. in a 1% aqueous solution. In the present invention, the viscosity of the hydroxypropylmethyl cellulose phthalate is determined by a B type viscosimeter. The hydroxypropylmethyl cellulose phthalate may be a commercially available product, such as HP-55, ex Shin-Etsu Chemical Co., Ltd.

The amount of the hydroxypropylmethyl cellulose phthalate in the aqueous dispersion is preferably from 0.5 to 15 parts by mass, more preferably from 0.2 to 5 parts by mass, relative to 100 parts by mass of the cosmetic pigment. If the amount is less than the aforesaid lower limit, the dispersibility of the pigment may be worse markedly, which is not preferred. If the amount is larger than the aforesaid upper limit, the viscosity of the aqueous dispersion may be significantly large, so that it is difficult to add the aqueous dispersion to a low-viscosity cosmetics and water repellency is poor.

Hydroxypropylmethyl cellulose acetate succinate may be used in combination with hydroxypropylmethyl cellulose phthalate. A total amount of hydroxypropylmethyl cellulose acetate succinate and hydroxypropylmethyl cellulose phthalate is preferably 0.1 to 10 parts by mass, per 100 parts by mass of the cosmetic pigment. The hydroxypropylmethyl cellulose acetate succinate preferably has a viscosity at 20° C. of from 1 to 200 mPa·s. For example, a commercially available product, Shin-Etsu AQOAT (ex Shin-Etsu Chemical Co., Ltd.) is usable.

The method for preparing the aqueous dispersion will be described below in detail.

The aqueous dispersion of the cosmetic pigment according to the present invention is obtained by a silylation of the cosmetic pigment with the alkyltrialkoxysilane in the presence of hydroxypropylmethyl cellulose phthalate in water. A pH of the reaction mixture is preferably basic. Sodium carbonate may be added to make a pH of the reaction mixture basic. For the reaction, an alkali catalyst is preferably added. Specifically, while stirring hydroxypropylmethyl cellulose phthalate and the cosmetic pigment in an ammonia solution with a stirrer such as homogenizer, an alkyltrialkoxysilane is added dropwise to cause silylation of the cosmetic pigment and to disperse the pigment in water. The reaction temperature may be adjusted properly and is preferably from room temperature to 80° C. Next, ammonia and a byproduct, ethanol, are distilled off, and the residue is neutralized with an acid such as citric acid to obtain an aqueous dispersion. The aqueous dispersion thus obtained preferably has a pH of from 6 to 8. If necessary, a step of breaking up aggregates with a wet pulverization device (e.g., Star Burst) may be carried out. An antiseptic agent or an antibiotic may be added to the aqueous dispersion thus obtained, if necessary.

The dispersion medium in the present aqueous dispersion is preferably water such as ion-exchanged water, purified water, distilled water and pure water and may contain an organic solvent if necessary. The surface of the cosmetic pigment obtained by the present preparation method is silylated and thereby hydrophobized and, therefore, aggregation of the cosmetic pigment is suppressed and dispersion stability in water improves. The silylation of the surface of the cosmetic pigment can be confirmed, for example, by a fact that a coating obtained has improved water repellency. The silylation can also be confirmed by observing the surface structure with a scanning electron microscope (SEM) or a transmission electron microscope (TEM).

The present invention also provides a liquid cosmetic comprising the aqueous dispersion. The amount of the aqueous dispersion of the present invention in the liquid cosmetic is preferably from 0.3 to 15 mass %, more preferably from 7 to 12 mass %, as the amount of the cosmetic pigment, based on the amount of the cosmetic. On account of incorporation of the aqueous dispersion in the aforesaid range, the liquid cosmetic has excellent long-term storage stability and gives a coating film having excellent water repellency. If the amount of the aqueous dispersion is less than the aforesaid lower limit, color development is poor and a liquid cosmetic is unsatisfactory. If the amount is larger than the aforesaid upper limit, the resulting cosmetic may have a too high viscosity, which is not preferred.

A film-forming polymer emulsion may be added to the liquid cosmetic of the present invention. The amount of the film-forming polymer emulsion is preferably from 5 to 25 mass % as a solid content (i.e., the amount of the film-forming polymer), based on the mass of the liquid cosmetic. By incorporating the film-forming polymer emulsion, the obtained liquid cosmetic such as eye liner is excellent in water resistance and durability so as to have make-up durable, for example, against sweat. Preferably, the amount of the film-forming polymer is from 5 to 15 mass %, more preferably from 7 to 12 mass %, relative to the amount of the liquid cosmetic. If the amount of the film-forming polymer is less than the aforesaid lower limit, the makeup coating has cracks, so that the liquid cosmetic does not function well. If the amount is larger than the aforesaid upper limit, a stress occurs while drying the makeup coating, so that an uncomfortable feeling remains on the skin.

The film-forming polymer emulsion to be used in the present invention may be any known one used in cosmetics. Examples of the film-forming polymer emulsion include emulsion of (co)polymeric resins composed of one or more monomers among acrylic acid, methacrylic acid, alkyl esters or derivatives thereof, styrene, and vinyl acetate. Specific examples of the resin emulsion include alkyl acrylate copolymer emulsion, alkyl methacrylate copolymer emulsion, (styrene/alkyl acrylate) copolymer emulsion, (styrene/alkyl methacrylate) copolymer emulsion, vinyl acetate polymer emulsion, (vinylpyrrolidone/styrene) copolymer emulsion, (alkyl acrylate/vinyl acetate) copolymer emulsion, (alkyl methacrylate/vinyl acetate) copolymer emulsion, (acrylic acid/alkyl acrylate)copolymer emulsion, (acrylic acid/alkyl methacrylate) copolymer emulsion, (methacrylic acid/alkyl acrylate) copolymer emulsion, (methacrylic acid/alkyl methacrylate) copolymer emulsion, and (alkyl acrylate/dimethicone) copolymer emulsion. In particular, emulsions of acrylic polymer composed of acrylic acid, methacrylic acid, or an alkyl ester or derivatives thereof are preferred.

The liquid cosmetic of the present invention may further comprise other components as long as its performance is not detracted. Examples of the other components include pH neutralizing agents, antiseptics, and thickeners. Further, the liquid cosmetic may comprise a polyhydric alcohol as a solvent, powders other than the surface-silylated cosmetic pigment, and a dispersant other than hydroxypropylmethyl cellulose phthalate.

Examples of the pH neutralizing agent include citric acid, ascorbic acid, sodium carbonate, and AMP (aminomethyl propanol). Examples of the antiseptic include phenoxyethanol, pentylene glycol, and ethanol. Examples of the thickener include carbomer, xanthan gum, and dextrin palmitate. The amount of them is not particularly limited and may be adjusted properly so that the effects of the present invention are not detracted.

The polyhydric alcohol is not particularly limited and may be any one used commonly in cosmetics. Examples of the polyhydric alcohol include glycol, propylene glycol, dipropylene glycol, 1,3-butylene glycol, and 1,2-pentylene glycol. These polyhydric alcohols may be used singly or two or more in combination. The amount of the glycol is preferably from 1 to 20 mass %, more preferably from 3 to 15 mass %, based on the total amount of the cosmetic.

The powders other than the surface-silylated cosmetic pigment are not particularly limited and may be any one used commonly in cosmetic. For example, they may have a shape and size, such as plate, spindle, or needle, or a particle structure such as porous or non-porous. Examples of the powder include inorganic powders, glitter powders, organic powders, pigment powders, and composite powders. More specific examples include inorganic powders such as Prussian blue, red oxide, yellow oxide, black iron oxide, aluminum oxide, cerium oxide, silicic anhydride, magnesium oxide, zirconium oxide, magnesium carbonate, calcium carbonate, chromium oxide, chromium hydroxide, carbon black, aluminum silicate, magnesium silicate, magnesium aluminum silicate, mica, synthetic mica, synthetic sericite, sericite, talc, kaolin, silicon carbide, barium sulfate, bentonite, smectite, and boron nitride; glitter powders such as bismuth oxychloride, iron oxide-coated mica, iron oxide-coated titanium mica, silicic acid anhydride-coated titanium mica, organic pigment-treated titanium mica, titanium oxide-coated glass powders, titanium oxide/iron oxide-coated glass powders, titanium oxide/silicic acid anhydride-coated glass powders, and aluminum powders; organic powders such as magnesium stearate, zinc stearate, N-acyl-lysine, and nylon; pigment powders such as organic tar-based pigments and lake pigments which are organic pigments, composite powders such as fine-particle titanium oxide-coated titanium mica, fine-particle zinc oxide-coated titanium mica, barium sulfate-coated titanium mica, titanium oxide-containing silicon dioxide, and zinc oxide-containing silicon dioxide; polyethylene terephthalate/aluminum/epoxy laminated powders, polyethylene terephthalate/polyolefin laminated film powders, and polyethylene terephthalate·polymethyl methacrylate laminated film powders. They may be used alone or in combination thereof.

As a dispersant other than hydroxypropylmethyl cellulose phthalate, preferred is those used mainly for dispersing the aforesaid powders other than the surface-silylated cosmetic pigment such as polyaspartic acid, polyacrylates, and water-soluble acrylic acid-based polymers and salts thereof.

The method for preparing the liquid cosmetic is not particularly limited and may be any known methods. For example, the aqueous dispersion of the present invention is put in a container and water (pure water or ion-exchanged water), a polyhydric alcohol (for example, glycol), a pH neutralizing agent, an antiseptic agent, a thickening agent and a film-forming agent are added with stirring by a known stirring means such as propeller type stirring device for a predetermined period of time. The stirring time may be set as desired, particularly stirring and mixing may be conducted for 30 minutes to 60 minutes.

The liquid cosmetic of the present invention may be put in a desired cosmetic container. The liquid cosmetic of the present invention preferably has a viscosity at 25° C. of 10 mPa·s or less, more preferably 2 to 10 mPa·s, still more preferably 4 to 7 mPa·s, as determined by a BM type viscometer (with No. 1 rotor, at 6 rpm). If the viscosity is larger than the aforesaid upper limit, it is difficult to constitute a spray type of cosmetic such as a sun screening agent. Alternatively, when the liquid cosmetic is put in a pen type container, the liquid cosmetic may not come out from a pen tip, which is not preferable.

Examples of the cosmetics comprising the aqueous dispersion of the present invention include sunscreen cosmetics, makeup cosmetics such as liquid foundation, eye liner, and mascara, and eye shadow, and hair cosmetics such as styling gel. The aqueous dispersion of the present invention does not cause caking and, therefore, it may be used not only in a liquid type, but also in a spray type. Further, it may be put in a pen type container.

### EXAMPLES

The present invention will be explained below in further detail with reference to a series of the Examples and the Comparative Examples, though the present invention is in no way limited by these Examples.

The cosmetic pigments used in the following Examples and Comparative Examples are as follows.

Titanium oxide 1: STR-100C (ex Sakai Chemical Industry Co.,Ltd., Al₂O₃-treated titanium oxide, average particle size: 16 nm)

Titanium oxide 2: MT-100AQ (ex TAYCA Co., Ltd., Sodium alginate-surface treated titanium oxide, average particle size: 15 nm)

Zinc oxide: FINEX-30W (ex Sakai Chemical Industry Co.,Ltd., average particle size: 35 nm)

Ultramarine: Ultramarine blue (ex Fujifilm Wako Pure Chemical Corporation, average particle size: from 3 to 5 μm)

Titanium mica: Timiron Super Red (ex Merck, average particle size: from 10 to 60 μm)

The other components used in the following Examples and Comparative Examples are as follows.

Hydroxypropylmethyl cellulose phthalate 1: (ex Shin-Etsu Chemical Co., Ltd., trade name: HP-55, viscosity at 20° C.: 40 mPa·s, as an aqueous 1% solution

Hydroxypropylmethyl cellulose phthalate 2: (ex Shin-Etsu Chemical Co., Ltd., trade name: HP-50, solution viscosity at 20° C.: 55 mPa·s (as an aqueous solution containing 1% of the ingredient)

Aqueous 30% solution of sodium polyaspartate: AQUADEW SPA-30 (ex Ajinomoto Healthy Supply Co., Inc.)

Carboxymethyl cellulose: Cellogen F—SB having a degree of etherification of 0.85 to 0.95, ex DKS Co. Ltd.

Methyltriethoxysilane: KBE-13 (ex Shin-Etsu Chemical Co., Ltd.)

### Example 1

### Preparation of Aqueous Dispersion 1 of Titanium Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed to prepare an aqueous 1 mass % solution of HP-55 in advance.

To 30 g of ion-exchanged water, 130 g of the aqueous 1 mass % solution of HP-55 was added, to which 70.2 g of titanium oxide, STR-100C (ex Sakai Chemical Industry Co.,Ltd.), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 297 g of an aqueous dispersion. 50 Grams of ion-exchanged water was used for washing. 12 Grams of an aqueous 10 mass % solution of citric acid was added to adjust the pH to 6.0 to 8.0. Thus obtained was aqueous dispersion 1 having a solid content of about 25 mass % (titanium oxide content: about 23 mass %). The aqueous dispersion 1 contained 0.4 mass % hydroxypropylmethyl cellulose phthalate, as calculated. According to observation by TEM, it was confirmed that the surface of the titanium oxide was silylated.

### Example 2

### Preparation of Aqueous Dispersion 2 of Titanium Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 30 g of ion-exchanged water, 200 g of the 1 mass % aqueous solution of HP-55 was added, to which 70.2 g of titanium oxide STR-100C (ex Sakai Chemical Industry Co.,Ltd.), 6.3 g of AQUADEW SPA-30 (ex Ajinomoto, sodium polyaspartate), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 313 g of an aqueous dispersion. 20 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 2 having a solid content of about 25 mass % (titanium oxide content: about 23 mass %). The aqueous dispersion 2 contained 0.6 mass % hydroxypropylmethyl cellulose phthalate, as calculated.

### Example 3

### Preparation of Aqueous Dispersion 3 of Titanium Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 30 g of ion-exchanged water, 200 g of the aqueous 1 mass % solution of HP-55 was added, to which 70.2 g of titanium oxide MT-100AQ (ex TAYCA Co., Ltd.), 6.3 g of AQUADEW SPA-30 (ex Ajinomoto, sodium polyaspartate), 53 g of ion-exchanged water, and 22.5 g of an aqueous 25% solution of ammonia were added, while stirring with a homomixer (number of revolutions: 5000 rpm). While stirring at 25° C., 4.5 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 313 g of an aqueous dispersion. 20 Grams of ion-exchanged water was used for washing. 12 Grams of a 10 mass % aqueous solution of citric acid was added to the resulting aqueous dispersion to neutralize the pH to 6.0 to 8.0 and thereto obtain an aqueous dispersion 3 having a solid content of about 25 mass % (titanium oxide content: about 23 mass %). The aqueous dispersion 3 contained 0.6 mass % hydroxypropylmethyl cellulose phthalate, as calculated.

### Example 4

### Preparation of Aqueous Dispersion 4 of Titanium Oxide

2 Grams of hydroxypropylmethyl cellulose phthalate (ex Shin-Etsu Chemical Co., Ltd., trade name: HP-50), 2 g of sodium carbonate, and 196 g of ion-exchanged water were mixed and dissolved to prepare an aqueous 1 mass % solution of HP-50 in advance.

To 30 g of ion-exchanged water, 130 g of the aqueous 1 mass % solution of HP-50 was added, to which 70.2 g of titanium oxide STR-100C (ex Sakai Chemical Industry Co.,Ltd.), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 297 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 4 having a solid content of about 25 mass % (titanium oxide content: about 23 mass %). The aqueous dispersion 4 contained 0.4 mass % hydroxypropylmethyl cellulose phthalate, as calculated.

### Example 5

### Preparation of Aqueous Dispersion 5 of Zinc Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 30 g of ion-exchanged water, 130 g of the aqueous 1 mass % solution of HP-55 was added, to which 70.2 g of zinc oxide FINEX-30W (ex Sakai Chemical Industry Co., Ltd.), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 297 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 5 having a solid content of about 25 mass % (zinc oxide: about 23 mass %). The aqueous dispersion 5 contained 0.4 mass % hydroxypropylmethyl cellulose phthalate, as calculated.

### Example 6

### Preparation of Aqueous Dispersion 6 of Ultramarine

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 30 g of ion-exchanged water, 130 g of the aqueous 1 mass % solution of HP-55 was added, to which 70.2 g of Ultramarine Blue (ex Fujifilm Wako Pure Chemical Corporation), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 297 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 6 having a solid content of about 25 mass % (ultramarine: about 23 mass %). The aqueous dispersion 6 contained 0.4 mass % hydroxypropylmethyl cellulose phthalate, as calculated.

### Example 7

Preparation of aqueous dispersion 7 of titanium mica 2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 30 g of ion-exchanged water, 130 g of the aqueous 1 mass % solution of HP-55 was added, to which 70.2 g of titanium mica, Timiron Super Red (ex Merck), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 297 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 7 having a solid content of about 25 mass % (titanium mica: about 23 mass %). The aqueous dispersion 7 contained 0.4 mass % hydroxypropylmethyl cellulose phthalate, as calculated.

### Comparative Example 1

Preparation of Aqueous Dispersion 8 of Titanium Oxide

To 155 g of ion-exchanged water, 70.2 g of titanium oxide STR-100C (ex Sakai Chemical Industry Co., Ltd.), 6.3 g of AQUADEW SPA-30 (ex Ajinomoto Healthy Supply, sodium polyaspartate), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 294 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 8 having a solid content of about 25 mass % (titanium oxide content: about 23 mass %).

### Comparative Example 2

Preparation of Aqueous Dispersion 9 of Titanium Oxide

2 Grams of Cellogen FS—B (ex DSK, carboxymethyl cellulose) and 198 g of ion-exchanged water were mixed and dissolved to prepare an aqueous 1 mass % solution of Cellogen FS—B in advance.

To 60 g of ion-exchanged water, 100 g of the aqueous 1 mass % solution of Cellogen FS—B was added, to which 70.2 g of titanium oxide STR-100C (ex Sakai Chemical Industry Co.,Ltd.), 53 g of ion-exchanged water, and 22.5 g of an aqueous 25% solution of ammonia were added, while stirring with a homomixer (number of revolutions: 5000 rpm). While stirring at 25° C., 9 g of methyltriethoxysilane was continuously added dropwise over 30 minutes to cause silylation and aged at an elevated temperature of 60° C. for one hour. Then, 80 ml of water containing ammonia and ethanol was distilled off in vacuum, while keeping the temperature at 60 to 70° C., to obtain about 292 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 9 having a solid content of about 25 mass % (titanium oxide content: about 23 mass %).

### Comparative Example 3

Preparation of Aqueous Dispersion 10 of Titanium Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 25 g of ion-exchanged water, 200 g of the aqueous 1 mass % solution of HP-55 was added, to which 70.2 g of titanium oxide STR-100C (ex Sakai Chemical Industry Co.,Ltd.) was added, while stirring with a homomixer (number of revolutions: 5000 rpm), to obtain about 274 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 10 having a solid content of about 25 mass % (titanium oxide content: about 23 mass %).

### Comparative Example 4

Preparation of Aqueous Dispersion 11 of Titanium Oxide

To 30 g of ion-exchanged water, 70.2 g of titanium oxide STR-100C (ex Sakai Chemical Industry Co.,Ltd.) and 200 g of ion-exchanged water were added, while stirring with a homomixer (number of revolutions: 5000 rpm). While stirring at 25° C., 53 g of ion-exchanged water, 9 g of methyltriethoxysilane and 22.5 g of an aqueous 25% solution of ammonia were continuously added dropwise over 30 minutes and aged at an elevated temperature of 60° C. for one hour. Then, 80 ml of water containing ammonia and ethanol was distilled off in vacuum, while keeping the temperature at 60 to 70° C., to obtain about 310 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 11 having a solid content of about 25 mass % (titanium oxide content: about 23 mass %).

### Comparative Example 5

Preparation of Aqueous Dispersion 12 of Zinc Oxide

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 25 g of ion-exchanged water, 200 g of the aqueous 1 mass % solution of HP-55 was added, to which 70.2 g of zinc oxide and FINEX-30W (ex Sakai Chemical Industry Co.,Ltd.) were added, while stirring with a homomixer (number of revolutions: 5000 rpm), to obtain about 274 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 12 having a solid content of about 25 mass % (titanium oxide content: about 23 mass %).

### Comparative Example 6

Preparation of Aqueous Dispersion 13 of Ultramarine

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 25 g of ion-exchanged water, 200 g of the aqueous 1 mass % solution of HP-55 was added, to which 70.2 g of Ultramarine Blue (ex Fujifilm Wako Pure Chemical Corporation) was added, while stirring with a homomixer (number of revolutions: 5000 rpm), to obtain about 274 g of an aqueous dispersion. 50 Grams of ion-exchanged water was used for washing. 12 Grams of an aqueous 10 mass % solution of citric acid was added to the resulting aqueous dispersion to neutralize the pH to 6.0 to 8.0 and thereto obtain an aqueous dispersion 13 having a solid content of about 25 mass % (titanium oxide content: about 23 mass %).

### Comparative Example 6

Preparation of Aqueous Dispersion 14 of Titanium Mica

2 Grams of hydroxypropylmethyl cellulose phthalate (ex Shin-Etsu Chemical Co., Ltd., trade name: HP-55), 2 g of sodium carbonate, and 196 g of ion-exchanged water were mixed and dissolved to prepare an aqueous 1 mass % solution of HP-55 in advance.

To 25 g of ion-exchanged water, 200 g of the aqueous 1 mass % solution of HP-55 was added, to which 70.2 g of titanium mica and Timiron Super Red (ex Merck) were added, while stirring with a homomixer (number of revolutions: 5000 rpm), to obtain about 274 g of an aqueous dispersion. 50 Grams of ion-exchanged water was used for washing. 12 Grams of an aqueous 10 mass % solution of citric neutralize acid was added to the resulting aqueous dispersion to the pH to 6.0 to 8.0 and thereto obtain an aqueous dispersion 14 having a solid content of about 25 mass % (titanium oxide content: about 23 mass %).

Evaluation of Water Repellency (or Water Resistance) of a Film

The aqueous dispersions 1 to 14 obtained in the Examples and Comparative Examples were each applied on a slide glass by a 2-mil bar coater, followed by drying at 25° C. for 2 hrs. to form a film. 0.1 Microliter of water was dropped on the resulting film. Thirty seconds after, a contact angle was determined with an automatic contact angle meter, DCA-VZ, ex Kyowa Interface Science. The results are shown in Tables 1 and 2.

### Storage Stability

The aqueous dispersion (100 ml) was put in a polyethylene bottle and left at 40° C. for 1 month. Then, the content was observed to know whether it was re-dispersible or not and whether caking occurred or not.

The results are as shown in Tables 1 and 2. When no precipitation remained to show good re-dispersion after shaking the bottle well, the dispersion was evaluated “good”. When precipitation was observed after shaking the bottle well, the dispersion was evaluated “caking”.

### Feeling in Touch

The feeling in touch of the aqueous dispersions was evaluated by a panel of 10 experts. A small portion of the aqueous dispersion was put on a fingertip and spread on the back of the hand to evaluate the feeling in touch, based on the following criteria.

Fresh, smooth, and comfortable without stickiness: 5 points

Smooth, but slightly rough: 3 points

Rough or sticky: 1 point

A total of the points given by the ten experts was calculated. When the total was 40 or more, the feeling in touch was evaluated as A. When the total was 15 or more and less than 40, the feeling in touch was evaluated as B. When the total was less than 15, the feeling in touch was evaluated as C. The results are as shown in Tables 1 and 2.

TABLE 1

| | | Example | | | | Comparative Example | | | |
|---|---|---|---|---|---|---|---|---|---|
| | | 1 | 2 | 3 | 4 | 1 | 2 | 3 | 4 |
| Aqueous dispersion No. | | 1 | 2 | 3 | 4 | 8 | 9 | 10 | 11 |
| Components | Titanium oxide1 | 100 | 100 | — | 100 | 100 | 100 | 100 | 100 |
| of the | Titanium oxide 2 | — | — | 100 | — | — | — | — | — |
| aqueous | Hydroxypropylmethyl | — | — | — | 1.8 | — | — | — | — |
| dispersion, | cellulose phthalate1 | | | | | | | | |
| part by mass | Hydroxypropylmethyl | 1.8 | 2.8 | 2.8 | — | — | — | 1.8 | — |
| | cellulose phthalate2 | | | | | | | | |
| | Sodium polyaspartate | — | 2.8 | 2.8 | — | 2.8 | — | — | — |
| | Thickener (CMC) for | — | — | — | — | — | 2.8 | — | — |
| | Comparison | | | | | | | | |
| | Methyltriethoxysilane | 12.8 | 12.8 | 6.4 | 12.8 | 12.8 | 12.8 | — | 12.8 |
| Evaluation | Solid content, % | 25.6 | 25.5 | 26.2 | 25.4 | 25.1 | 25.8 | 25.5 | 25.6 |
| | pH | 7.8 | 7.4 | 7.8 | 7.5 | 7.4 | 7.3 | 7.5 | 7.6 |
| | Water repellency, | 94 | 88 | 71 | 88 | 28 | <10 | <10 | <10 |
| | water contact angle, ° | | | | | | | | |
| | Storage stability, | good | good | good | good | caking | caking | caking | caking |
| | 40° C. for one month | | | | | | | | |
| | Feeling in touch | A | A | A | A | A | A | A | C |

TABLE 2

| | | Example | | | Comparative Example | | |
|---|---|---|---|---|---|---|---|
| | | 5 | 6 | 7 | 5 | 6 | 7 |
| Aqueous dispersion No. | | 5 | 6 | 7 | 12 | 13 | 14 |
| Components of the aqueous | Zinc Oxide | 100 | — | — | 100 | — | — |
| dispersion, | Ultramarine | — | 100 | — | — | 100 | — |
| part by mass | Mica titanium | — | — | 100 | — | — | 100 |
| | Hydroxypropylmethyl | 1.8 | 1.8 | 1.8 | 1.8 | 1.8 | 1.8 |
| | cellulose phthalate 2 | | | | | | |
| | Methyltriethoxysilane | 12.8 | 12.8 | 12.8 | — | — | — |
| Evaluation | Solid content, % | 25.3 | 25.4 | 25.4 | 25.2 | 25.5 | 25.6 |
| | pH | 7.7 | 7.7 | 7.5 | 7.6 | 7.3 | 7.8 |
| | Water repellency, | 94 | 90 | 89 | <10 | <10 | <10 |
| | water contact angle, ° | | | | | | |
| | Storage stability, | good | good | good | caking | caking | caking |
| | 40° C. for one month | | | | | | |
| | Feeling in touch | A | A | A | A | A | A |

As seen in Tables 1 and 2, the aqueous dispersion of Comparative Example 1 comprising, as a dispersant for titanium oxide, sodium polyaspartate instead of hydroxypropylmethyl cellulose phthalate gave a poor water repellency in a film and caused caking with time. This aqueous dispersion was inferior in dispersion stability.

The aqueous dispersion of Comparative Example 2 comprising Cellogen F—SB as a thickening agent also gave a poor water repellency in a film and was inferior in dispersion stability.

As seen in Comparative Examples 3 and 5 to 7, the aqueous dispersions comprising titanium oxide, zinc oxide, ultramarine, or titanium mica, respectively, which were not surface-treated with methyltriethoxysilane, also gave a poor water repellency in a film and was inferior in dispersion stability.

Further, the aqueous dispersion of Comparative Example 4 comprising no hydroxypropylmethyl cellulose phthalate or other dispersant also gave a poor water in a film repellency, was inferior in dispersion stability, and had poor feeling in touch.

In contrast, as seen in Examples 1 to 7 in Tables 1 and 2, the aqueous dispersions of the present invention gave a film having good water repellency, was excellent in dispersion stability, and had good feeling in touch.

### INDUSTRIAL APPLICABILITY

The aqueous dispersion of the present invention is excellent in dispersion stability. The aqueous dispersion is suitable for cosmetics because it provides a film which is excellent in water resistance and excellent in storage stability.

1. An aqueous dispersion comprising a cosmetic pigment, water and hydroxypropylmethyl cellulose phthalate, wherein the cosmetic pigment is surface-silylated with an alkyltrialkoxysilane.

2. The aqueous dispersion according to claim 1, wherein the cosmetic pigment is at least one selected from the group consisting of titanium oxide, zinc oxide, ultramarine, and titanium mica.

3. The aqueous dispersion according to claim 1, wherein an amount of hydroxypropylmethyl cellulose phthalate is 0.5 to 15 parts by mass and an amount of the alkyltrialkoxysilane is 0.1 to 20 parts by mass, relative to 100 parts by mass of the cosmetic pigment.

4. A method for preparing an aqueous dispersion wherein the method comprises a step of reacting a cosmetic pigment with alkyltrialkoxysilane in water containing hydroxypropylmethyl cellulose phthalate to obtain the aqueous dispersion of a cosmetic pigment surface-silylated with the alkyltrialkoxysilane.

5. The method according to claim 4, wherein cosmetic pigment is at least one selected from the group consisting of titanium oxide, zinc oxide, ultramarine, and titanium mica.

6. The method according to claim 4, wherein the aqueous dispersion comprises the surface-silylated cosmetic pigment in an amount of 1 to 30 mass % as the pigment relative to the aqueous dispersion, and comprises 0.5 to 15 parts by mass of the hydroxypropylmethyl cellulose phthalate and 0.1 to 20 parts by mass of the alkyltrialkoxysilane, relative to 100 parts by mass of the cosmetic pigment.

7. A liquid cosmetic comprising the aqueous dispersion according to claim 1, wherein the liquid cosmetic comprises 0.3 to 15 mass % of the cosmetic pigment, relative to the liquid cosmetic.
